(19) United States
(12) Patent Application Publication
(10) Pub. No.: US 2025/0204990 A1
(43) Pub. Date: Jun. 26, 2025

(54) SYSTEM AND METHOD FOR IMAGE-GUIDED ENDOLUMINAL INTERVENTION USING REGISTERED EXTENDED REALITY VISUALIZATION

(71) Applicant: MEDIVIEW XR, INC., Cleveland, OH (US)

(72) Inventors: Jeffrey Harold Yanof, Solon, OH (US); Gaurav Gadodia, Chicago, IL (US); Peter Nicholas Braido, Wyoming, MN (US); Aydan Thomas Hanlon, Akron, OH (US); Ross Daniel Hinrichsen, Plymouth, MN (US); Adam E. Rakestraw, Findlay, OH (US)

(21) Appl. No.: 18/980,535

(22) Filed: Dec. 13, 2024

Related U.S. Application Data
(60) Provisional application No. 63/613,989, filed on Dec. 22, 2023.

(51) Int. Cl. A61B 34/20; A61B 8/00; A61B 8/08; A61B 8/12

(52) U.S. Cl. CPC A61B 34/20; A61B 8/0841; A61B 8/0891; A61B 8/12; A61B 8/4254; A61B 8/445; A61B 8/463; A61B 8/5261; A61B 2034/2063

(57) ABSTRACT

A system for image-guided intervention of a patient is provided. The system can include an endoluminal probe, a tracking system, a display system, and a registration system. The endoluminal probe can be configured to acquire a real-time image of an internal site within the patient. The tracking system can be configured to track a position and an orientation of the endoluminal probe. The display system can be configured to display the real-time image acquired by the endoluminal probe. The registration system can be configured to register the real-time image from the endoluminal probe with a preoperative image of the internal site. The display system can overlay the registered real-time image from the endoluminal probe with the preoperative image to assist in navigation and intervention at the internal site.

### CROSS-REFERENCE TO RELATED APPLICATIONS

This application claims the benefit of U.S. Provisional Application No. 63/613,989, filed on Dec. 22, 2023. The entire disclosure of the above application is incorporated herein by reference.

### FIELD

The present technology relates to holographic augmented or extended reality medical applications and, more particularly, endoluminal systems and methods employing holographic extended reality and temporal registration.

### INTRODUCTION

This section provides background information related to the present disclosure which is not necessarily prior art.

Image-guided surgery can be utilized for a variety of different procedures. Image-guided surgery can visually correlate intraoperative data with preoperative and postoperative data to aid a practitioner. The use of image-guided surgery has been shown to increase the success of a procedure. Image-guided surgery can be further enhanced through the use of augmented reality (AR) technology, also referred to as extended reality. AR is an interactive experience of a real-world environment where one or more features that reside in the real world are enhanced by computer-generated perceptual information, sometimes across multiple sensory modalities. In a medical setting, AR technology can be useful for enhancing the real environment in the patient care setting. For example, a practitioner can view content-specific information in the same field of view of the patient while performing a medical procedure, without having to change their gaze during the medical procedure.

Various image-guided interventions rely on accurate localization of an instrument and visualization of the anatomy of the patient to facilitate an effective procedure. While a preoperative image provides valuable anatomical context, a preoperative image does not reflect intraoperative change. A practitioner can try to mentally map live endoscopic video onto the preoperative image, but mental mapping can result in a lack of precision which is undesirable in a medical procedure. Additionally, endoscope tracking methods alone provide only external or limited localization. Electromagnetic (EM) and optical systems track rigid instruments but cannot see inside the patient. Intraoperative imaging, like ultrasound and fluoroscopy, visualizes internal anatomy but lacks wider context. A practitioner must constantly reference a screen and mentally integrate spatial data, increasing cognitive load on the practitioner during the procedure.

AR systems have attempted to address these challenges. By overlaying live video onto a medical image, AR can intuitively integrate external and internal views. However, the AR system can lack robust tracking and registration methods to accurately align an endoscopic tool with patient anatomy. Additional technical challenges have also hindered AR adoption for endoscopy. Solid organ deformation, respiratory motion, tissue resection and insufflation can all cause anatomy to deviate from preoperative imaging. Surface registration techniques like point clouds cannot model these complex changes and internal reference points are needed for accurate alignment. Probe tracking methods also lack robustness and precision inside the body. External navigation suffers interference, while internal EM sensors cover limited fields of view.

A system is needed to provide robust, localized registration between real-time endoluminal imaging and surgical anatomy. By accurately tracking flexible probes and mapping surrounding tissue changes, such a technology could maintain a high-fidelity augmented view, which could improve navigation, reduce cognitive load, and enhance training for endoscopic intervention. There is accordingly a continuing need for robust and localized registration between real-time endoluminal imaging and patient anatomy during a medical intervention, where it would be desirable to provide three dimensional (3D) holographic visualization of data that is not possible in two dimensions (2D).

### SUMMARY

In concordance with the instant disclosure, systems and methods have surprisingly been discovered that solve the above-mentioned problems through accurate endoluminal probe tracking and real-time registration of probe images with surgical anatomy, and by providing a dynamically updated augmented view aligned to localized tissue, and which provide for enhanced navigation and reduced cognitive burden during complex endoscopic interventions.

The present technology includes articles of manufacture, systems, and processes that relate to real-time image-guided endoluminal intervention using augmented reality (AR). Ways are provided to use a tracking-enabled endoluminal imaging probe, a registration system aligning a real-time endoluminal video feed, ultrasound imaging with a three-dimensional anatomical model, and a wearable display device presenting unified visualizations fusing endoscopic perspectives with an anatomy reconstruction and an instrument position to guide a minimally invasive procedure. The present technology facilitates endoluminal navigation, device guidance, spatial understanding, along with pre- and post-operative analytics to improve efficiency, targeting accuracy, and procedural outcome.

The present technology can further include registering an image from the endoluminal probe to the patient. A preoperative image (e.g., CT) can also be registered thereto. The registered images can provide three-dimensional imaging that is stereoscopically projected through the augmented reality system. Data from the patient (e.g., ECG) can be used to synchronize the animation from the patient to animate the registered image, including CT data, for example, thereby providing an animated anatomical model. In this way, the physical anatomy is synchronized and updated based upon the patient and provides an accurate and real-time augmented reality representation to improve a practitioner's interaction with the patient anatomy.

The present technology can further include a system and method for image-guided intervention using an endoluminal probe with registered extended reality (XR) visualization. The system can be configured for tracking the position of an endoluminal probe, acquiring a real-time endoluminal image from the probe, registering the real-time image with a preoperative image, and displaying the registered image in an extended reality headset to assist navigation and intervention. The endoluminal probe can be configured to acquire a real-time image of an internal site within a patient. A tracking system tracks the position and orientation of the endoluminal probe. A display system can display the real-time endoluminal image and can overlay the real-time endoluminal image with a preoperative image, after the image is registered by a registration system. The augmented display assists the practitioner in navigating to the target anatomy and performing the intervention with a catheter or other suitable instrument. The tracking system can include inertial, electromagnetic, optical, fiber optic shape sensing, and/or other sensors integrated with the probe. The preoperative image can be from CT, MRI, PET or other modalities. Tracking can include various tracking systems, including near infrared (NIR) and laser (e.g., light detection and ranging (LIDAR)) for range finding for anatomical positioning.

A method can include steps of inserting the endoluminal probe, tracking the position of the endoluminal probe, acquiring a real-time endoluminal image, registering the endoluminal images with a preoperative image of the anatomy, and displaying the registered, overlaid image in an XR headset to assist surgical navigation and intervention. The registration can be dynamically updated to account for patient motion. Various endoluminal probes can be employed, as well as an intravascular ultrasound catheter, a transesophageal echocardiogram probe, or an intracardiac echocardiography catheter. Additional instruments such as guidewires, needles or catheters can also be tracked and displayed in the augmented endoluminal view.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a schematic of a system for image-guided intervention of a patient;

FIG. 2 is a schematic of the system for image-guided intervention of a patient;

FIG. 3 is a flowchart depicting a method for image-guided intervention of a patient;

FIG. 4 is a flowchart depicting the method for image-guided intervention of a patient;

FIGS. 5A-5B provide top and bottom perspective views of an EM sensor to be used with the system for image-guided intervention of a patient for registration of a 3D retrospective tomographic image;

FIG. 6 is a depiction of anatomy involved in a transjugular intrahepatic portosystemic shunt (TIPS) procedure that can be performed using the system for image-guided intervention of a patient of FIG. 1;

FIGS. 7A-7B are images of intravascular ultrasound (IVUS) for atherosclerosis applications;

FIGS. 8A-8D are images of intracardiac echocardiography (ICE) for cardiac ablation applications; and

FIG. 9 is an image of endoluminal ultrasound (ELUS) for urothelial or bladder carcinoma applications.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as can be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items can be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that can arise from ordinary methods of measuring or using such parameters.

Although the open-ended term “comprising,” as a synonym of non-restrictive terms such as including, containing, or having, is used herein to describe and claim embodiments of the present technology, embodiments can alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that can be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter can define endpoints for a range of values that can be claimed for the parameter. For example, if Parameter X is exemplified herein to have value A and also exemplified to have value Z, it is envisioned that Parameter X can have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X can have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it can be directly on, engaged, connected or coupled to the other element or layer, or intervening elements or layers can be present. In contrast, when an element is referred to as being “directly on,” “directly engaged to,” “directly connected to” or “directly coupled to” another element or layer, there can be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. can be used herein to describe various elements, components, regions, layers and/or sections, these elements, components, regions, layers and/or sections should not be limited by these terms. These terms can be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, can be used herein for ease of description to describe one element or feature's relationship to another element(s) or feature(s) as illustrated in the figures. Spatially relative terms can be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

As used herein, the term “interventional device” or “tracked instrument” refers to a medical instrument used during the medical procedure.

As used herein, the term “tracking system” refers to something used to observe one or more objects undergoing motion and supply a timely ordered sequence of tracking data (e.g., location data, orientation data, or the like) in a tracking coordinate system for further processing. As an example, the tracking system can be an electromagnetic, and optical (e.g., fiber optic) tracking system that can observe an interventional device equipped with a sensor-coil as the interventional device enters, moves through, exits, and while outside of a patient's body.

As used herein, the term “tracking data” refers to information recorded by the tracking system related to an observation of one or more objects undergoing motion.

As used herein, the term “head-mounted device” or “headset” or “HMD” refers to a display device, configured to be worn on the head, that has one or more display optics (including lenses) in front of one or more eyes. These terms can be referred to even more generally by the term “augmented reality system,” although it should be appreciated that the term “augmented reality system” is not limited to display devices configured to be worn on the head.

In some instances, the head-mounted device can also include a non-transitory memory and a processing unit. An example of a suitable head-mounted device is a Microsoft HoloLens®.

As used herein, the term “imaging system,” “image acquisition apparatus,” “image acquisition system” or the like refer to technology that creates a visual representation of the interior of a body of a patient. For example, the imaging system can be a computed tomography (CT) system, a fluoroscopy system, positron emission computed tomography, magnetic resonance imaging (MRI) system, an ultrasound (US) system including contrast agents and color flow doppler, or the like.

As used herein, the term “coordinate system” or “augmented realty system coordinate system” refer to a 3D Cartesian coordinate system that uses one or more numbers to determine the position of points or other geometric elements unique to the particular augmented reality system or image acquisition system to which it pertains. For example, 3D points in a headset coordinate system can be translated, rotated, scaled, or the like, from a standard 3D Cartesian coordinate system.

As used herein, the term “image data” or “image dataset” or “imaging data” refers to information recorded in 3D by the imaging system related to an observation of the interior of the body of the patient. For example, the “image data” or “image dataset” can include processed two-dimensional (2D) or three-dimensional (3D) images or models such as tomographic images, e.g., represented by data formatted according to the Digital Imaging and Communications in Medicine (DICOM) standard or other relevant imaging standards.

As used herein, the term “imaging coordinate system” or “image acquisition system coordinate system” refers to a 3D Cartesian coordinate system that uses one or more numbers to determine the position of points or other geometric elements unique to the particular imaging system. For example, 3D points and vectors in the imaging coordinate system can be translated, rotated, scaled, or the like, to the Augmented Reality system (head mounted displays) 3D Cartesian coordinate system.

As used herein, the term “hologram”, “holographic,” “holographic projection”, or “holographic representation” refer to a computer-generated image stereoscopically projected through the lenses of a headset. Generally, a hologram can be generated synthetically (in an augmented reality (AR)) and is not a physical entity.

As used herein, the term “physical” refers to something real. Something that is physical is not holographic (or not computer-generated).

As used herein, the term “two-dimensional” or “2D” refers to something represented in two physical dimensions.

As used herein, the term “three-dimensional” or “3D” refers to something represented in three physical dimensions. An element that is “4D” (e.g., 3D plus a time and/or motion dimension) would be encompassed by the definition of three-dimensional or 3D.

As used herein, the term “integrated” can refer to two or more things being linked or coordinated. For example, a coil-sensor can be integrated with an interventional device.

As used herein, the term “real-time” or “near-real time” or “live” refers to the actual time during which a process or event occurs. In other words, a real-time event is done live (within milliseconds so that results are available immediately as feedback). For example, a real-time event can be represented within 100 milliseconds of the event occurring.

As used herein, the terms “subject” and “patient” can be used interchangeably and refer to any vertebrate organism.

As used herein, the term spatial “registration” refers to steps of transforming tracking and imaging data associated with virtual representation of tracked devices—including holographic guides, applicators, and ultrasound image stream—and additional body image data for mutual alignment and correspondence of said virtual devices and image data in the head mounted displays coordinate system enabling a stereoscopic holographic projection display of images and information relative to a body of a physical patient during a procedure, for example. Registration systems and processes are further described in U.S. Patent Application Publication No. 2018/0303563 to West et al. and applicant's co-owned U.S. patent application Ser. No. 17/110,991 to Black et al. and U.S. patent application Ser. No. 17/117,841 to Martin III et al., the entire disclosures of which are incorporated herein by reference. The present technology can further include registration components and operations as provided by the augmented reality systems with improved registration methods as well as methods for multi-therapeutic deliveries disclosed in applicant's co-owned U.S. Patent Application Ser. No. 63/504,675 to Hanlon et al., which is incorporated herein by reference.

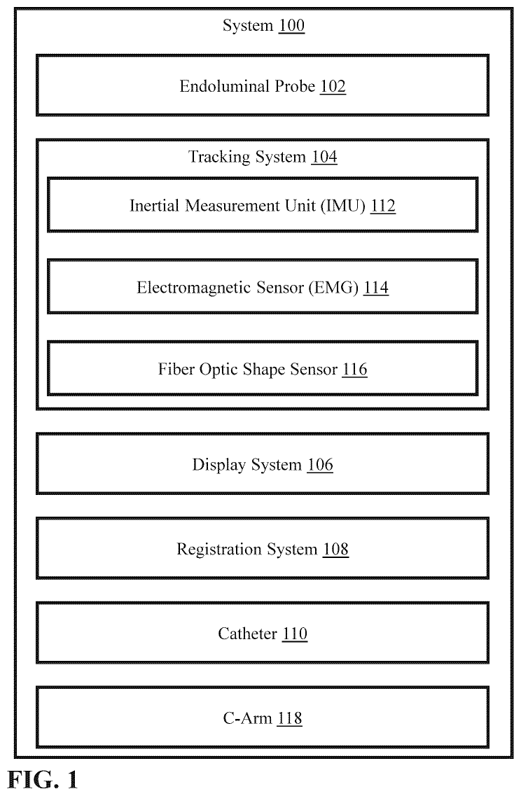

The present technology relates to systems and methods for image-guided endoluminal intervention using registered extended reality visualization, as shown and described in FIGS. 1-4 hereinbelow. The present technology improves image-guided endoluminal intervention by unifying real-time endoscopic imaging, a three-dimensional anatomical model from preoperative scanning, instrument tracking data, and advanced visualization to enhance navigation, spatial understanding, and procedural efficiency. In this way, improved navigation, reduced practitioner cognitive load, and enhanced training for endoscopic interventions are provided. An internal reference point for high-fidelity alignment of a flexible probe or for modeling a complex anatomical deformation can also be provided for various endoluminal applications.

A system for image-guided intervention of a patient can include an endoluminal probe, a tracking system, a display system, and a registration system. The endoluminal probe can be configured to acquire a real-time image of an internal site within the patient. The tracking system can be configured to track a position and an orientation of the endoluminal probe. The display system can be configured to display the real-time image acquired by the endoluminal probe. The registration system can be configured to register the real-time image from the endoluminal probe with a preoperative image of the internal site. In this way, the display system can overlay the registered real-time image from the endoluminal probe with the preoperative image to assist in navigation and intervention at the internal site.

A method for image-guided intervention of a patient can include the following steps. An endoluminal probe can be inserted internally to acquire a real-time image of an internal site within the patient. A position and an orientation of the endoluminal probe can be tracked. The real-time image from the endoluminal probe can be displayed on a display system. The real-time image can be registered with a preoperative image of the internal site. The registered real-time images can be overlayed on the preoperative image to assist in navigation and intervention of the internal site.

The system of the present disclosure can be employed to facilitate the method described herein. Generally, the method can include a pre-operative component, a spatial registration component, an intra-operative navigation component, and a post-operative component. The pre-operative component can include collecting patient data via a scanning device such as an MDCT, CBCT, or PET, as desired. The spatial registration component can include using an advanced modeling target (AMT) to transform the data collected by the AMT to a headset coordinate system and therefore to a headset itself. The intra-operative navigation can include utilizing the tracking system, which can include an ultrasound and EM navigation system. The ultrasound can include the endoluminal probe and the EM navigation system can include a catheter. The tracking system can work to replay the position of the endoluminal probe and the catheter to the headset. A trajectory can be planned for the catheter and the practitioner can adjust the catheter as it is tracked intraoperatively to effectively perform the procedure. The post-procedural component can allow for analysis of a biopsied area as well as the planned trajectory itself.

Turning now to the probe of the system, which can include an endoluminal probe. The probe can include a medical imaging device that uses ultrasound technology to help the practitioner see inside the body during the procedure. As the probe moves, it sweeps a sheet of light across tissue surfaces and rapidly moves back and forth along the scanning path. The movement allows the system to create a near real-time, live 3D view of the tissue being examined.

The system can have various types of ultrasound probes, including a rigid external probe, a rigid internal probe, and a flexible internal probe. With respect to an endoluminal procedure, the flexible internal probe can be utilized to navigate the internal anatomy of the patient. In other embodiments, the probe can include a transesophageal echocardiography (TEE) probe for heart imaging, an intravascular ultrasound (IVUS) probe for looking inside blood vessels, and an intra-cardiac echocardiography (ICE) probe that can be moved around inside heart chambers, as examples. A skilled artisan can select a suitable probe within the scope of the present disclosure.

The probe can include tracking technology to aid in monitoring the exact position and movement of the probe while in use. The tracking technology can include a magnetic sensor embedded in a tip of the probe that works with an electromagnetic field generator, a fiber optic sensor that runs along the length of the probe to detect the shape of the probe, and/or an inertial measurement unit (IMU) that tracks the orientation and the movement of the probe. It should be appreciated that the tracking system can use any type of sensor such as an EM sensor, IMU, optical sensor, IR sensor, acoustic sensor, and combinations thereof.

Turning now to the tracking system, the tracking system can combine multiple technologies to provide real-time monitoring of a medical device during a procedure. The system can use at least three tracking approaches that can work independently or together including EM tracking, fiber optic shape sensing, and/or advanced model targeting. First, EM tracking can use miniaturized sensors embedded in the probe to generate an electrical current when placed within an electromagnetic field created by a transmitter unit. This allows for real-time 3D tracking of the probe without requiring direct line of sight. The system can be integrated with fluoroscopy imaging by installing the transmitter unit within the fluoroscopy detector, enabling automatic registration between the tracking and imaging system. Second, fiber optic shape sensing can provide tracking capabilities that are immune to electromagnetic interference. The system can employ optical fibers embedded along the length of the probe to detect the exact shape and position of the probe. In one embodiment, the fiber optic component can include a sensing length that can be customized via both the length of the fiber optic component and the number of sensors on the fiber optic component. For example, the fiber optic component can include 8 sensors per cable, with each sensor containing 4 fibers each. A skilled artisan can select a suitable configuration within the scope of the present disclosure. Third, AMT can be combined with a wireless IMU to provide further tracking capability. The AMT can use computer vision to recognize and track the probe visually, while the IMU provides orientation and movement data. The combination can be useful when tracking a device through varying conditions and lighting environments.

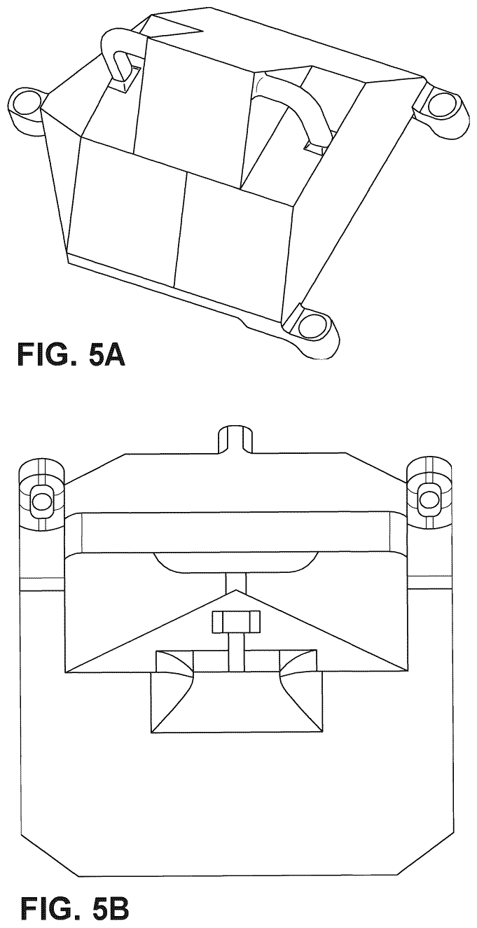

It should be noted that an exemplary embodiment of the EM sensor is shown in FIGS. 5A-5B. It should be recognized that a LIDAR camera in the AR headset and/or optical sensors can capture and also map the AMT or image target model, the surface of the body, and the contact of the feet of the sensor. In addition to the EM sensor, other sensors can be used having a reflective infrared disc, ball, and/or a battery powered LED at various other locations on the patient to track and register the model.

Referring now to spatial registration, it should be appreciated that AMT combined with an IMU via Bluetooth or radiofrequency and/or AMT combined with wireless EM tracking can militate against the need for a wired/tethered navigation modality that require set up during the workflow. In this way, AMT does not require a fixed location for applications such as point of care ultrasound. Advanced model targeting can include an entire 3D model, where it is also possible to use one or more 2D image targets to create a 3D reference, which can standalone but can also be paired with any of the sensors and sensing methods described herein.

In the case of mobile wireless EM and ultrasound, it can be beneficial for quick setup to have the EM field denoted in 3D on the AR headset to ensure the patient and device tracking is infield of optimal placement. A 3D AR representation of a transmitter and a receiver space with any interference within can provide for an efficient workflow.

AMT or IMU combined with fiberoptic can use a tethered system, however, is not affected by electromagnetic fields so the combination can be used in an environment that uses multi-detector row computed tomography (MDCT), for example. Note that AMT can aid in redundant positioning of the external part of the device in the event of EM interference and especially with respect to a rigid probe. For example, the catheter can be fixed to a rigid robot, where the position of the device origin may need to be localized in virtual space using model targeting. If needed, redundancy can be built in with model targeting, fiber optics and IMU.

AMT combined with wireless EM and wireless IMU can militate against the need for a wired or tethered navigation modality that requires set up during the workflow. In an example of an endocavitary probe where the 3D model is localized by the headset cameras with line of sight independent of EM field distortion, the EM sensor can give positional and orientation data most accurately but with range and interference limitations, while the hybridization of the IMU to track change in location from a distance as a redundant cross-referencing set of sensors can provide an optimum experience. Together the EM sensor and IMU can fully define the position and orientation of the device and associated imaging fusion over a wide range of distances and interferences for efficient everchanging use conditions and workflows.

AMT combined with wireless optical and wireless IMU can militate against the need for a wired or tethered navigation modality that requires set up during the workflow. An optical sensor, such as active or passive infrared signals, can be transmitted/received via a fixed, standalone camera or optical sensor attached to and in communication with the augmented reality (AR) headset. Together, the AMT, wireless optical, and wireless IMU can define the position and orientation of the device and associated imaging fusion.

Any number of combinations can be included to optimize navigation for a particular situation via real time redundant or complimentary sub-systems. When integrated into the overall system, the registration system not only enhances the accuracy and reliability of the output, but also affords the versatility to interchange sub-systems as needed for differing use cases, e.g., TIPS vs cardiovascular.

The system can use the tracking method through a registration process that aligns three coordinate systems: a head mounted display (HMD) coordinate system, a CT coordinate system (from medical imaging), and an EM tracking coordinate system. The integration of the three coordinate systems allows all the tracking data to be combined and displayed in a unified view. For practical implementation, the system can display the tracking information in several ways. For example, the system can show the EM field boundaries in augmented reality to help a practitioner position a patient optimally, visualize device trajectory in real-time, and overlay tracking information onto live ultrasound or fluoroscopy images. It should be noted that the system can be particularly applicable in a procedure like TIPS, where the system can track both the ultrasound probe and catheter simultaneously, helping guide the procedure more accurately.

The display system of the system can include AR technology through a head-mounted display (HMD) to create an enhanced visualization of the surgical field. The system works by projecting a stereoscopic image based on 3D medical imaging that is augmented onto the physical patient through the see-through head mounted display. The image is correlated with the actual orientation of the patient. For example, when displaying a sagittal section, the sagittal section can be projected with head-foot and anterior-posterior axes that match the actual anatomy of the patient, rather than showing them on a traditional 2D monitor.

The display system can also integrate real-time tracking information with pre-operative imaging. For example, during a TIPS procedure, the display system can show the position of surgical instruments in relation to both live ultrasound images and pre-operative CT scans, which can assist the practitioner with navigating complex procedures with greater precision. The system can also automatically adjust the display based on where the practitioner is looking, toggling between different types of information as needed. To manage the large amount of data being displayed, the system can selectively show smaller portions of the field of view or target specific areas thereby reducing the amount of data being transmitted and displayed, while still maintaining sufficient spatial and temporal resolution for accurate surgical navigation. The display can also automatically turn on and off depending on where the surgeon directs their view, helping to manage cognitive load during a procedure.

Turning now to the registration system, which can align the HMD coordinate system, the CT coordinate system, and the EM tracking coordinate system to create a merged view of the surgical field. The system can achieve alignment by establishing spatial relationships between different objects, transforming each from its own independent model coordinate system into a common global coordinate system. To merge the systems, the registration process uses a combination of spatial mapping and point-based registration. For example, registration can be performed using at least three or more points that can be superimposed or fused into a common object coordinate system for virtual data and live data. The registration process can also use surface matching, where a virtual surface is aligned with a live surface of the patient, or shape matching, where a virtual 3D shape is aligned with a live shape, which allows for precise alignment of pre-operative imaging with the actual patient anatomy during the procedure.

In certain embodiments, the system can use optical markers, which can include different geometric shapes, patterns, QR codes, bar codes, or alphanumeric codes, to help maintain accurate registration throughout the procedure, as desired. The system can also compensate for patient movement and breathing. Ultrasound and CT data can be phase gated during a cardiac cycle. Respiratory motion can be tracked with the AMT separately or in combination of the other mentioned sensors, as well as confirming the reproduction of the breath hold when scanning. A resemblance of a beating heart can be shown in AR relative to the delivery system and probe as the practitioner navigates in operation. For example, illustrating a phased 4D representation of the beating heart, for example, combining spatial to temporal mechanical changes can be based on strain calculation from ultrasound via electromechanical wave imaging. The 4D gated representation can be registered to the ultrasound probe.

Turning now to the catheter of the system. The catheter can be inserted through the anatomy of the patient to allow access to an internal site through a blood vessel or other natural passage. The catheter can include tracking capability, including a fiber optic shape sensor, as described herein. It should be noted that the catheter can incorporate various types of sensors, including an IMU, an EM sensor, and a fiber optic shape sensor, which work together to provide tracking of the catheter throughout the procedure.

For specific procedures like transjugular intrahepatic portosystemic shunt (TIPS), the catheter can be inserted through the jugular vein and guided into the hepatic vasculature. The catheter can be equipped with an intravascular ultrasound (IVUS) capability at a tip of the catheter, allowing the catheter to capture real-time images of the surrounding vessels and anatomy as the catheter moves through the body.

For a procedure requiring an additional instrument, the catheter can serve as a guide for other tools, such as a puncture needle or stent delivery system. The tracking system can be configured to monitor both the catheter and the additional instrument, displaying the relative positions and projected trajectories to assist in precise navigation and intervention.

The catheter also allows for the integration of a wireless sensor, which can help reduce procedural complexity by eliminating the need for wired connections while maintaining accurate tracking capabilities. The wireless functionality can be beneficial in maintaining a streamlined workflow during an interventional procedure.

The endoluminal surgical system can find use in various examples of services, conditions, and end-users. The system can be used across multiple service types and conditions in different healthcare settings. For laparoscopic surgery, applications include appendectomy, hysterectomy, myomectomy, colectomy and other procedures. The system also supports robotic surgery applications including colorectal surgery, gynecologic surgery and other robotic surgical procedures. In terms of medical conditions, the system can be utilized for gastrointestinal conditions, spinal conditions, gynecologic conditions, and other medical conditions requiring surgical intervention. The end users of this system include both hospitals and clinics as well as ambulatory surgical centers.

It should be appreciated that the medical imaging system improves image quality, ease of use, and portability. The present navigation system improves and, in some cases, facilitates the merging of multiple modalities like EM and impedance-based sensor navigation. The system can be utilized with different methods of therapy delivery (e.g., microwave, radiofrequency, cryoablation, irreversible electroporation, pulsed electric field, etc.).

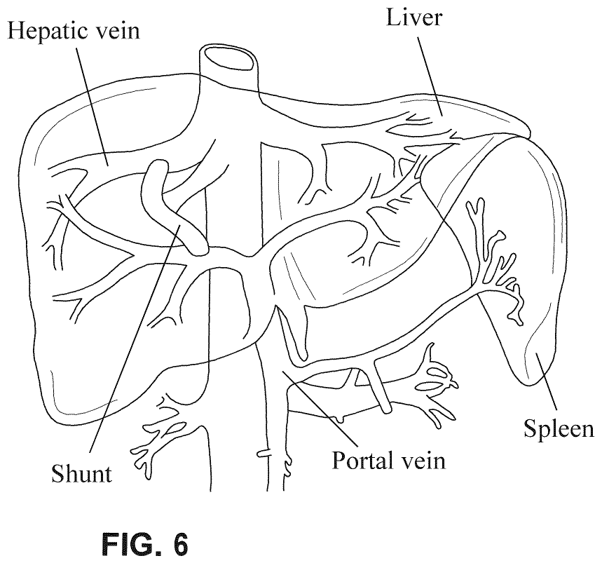

One application of the present system relates to the Transjugular Intrahepatic Portosystemic Shunt (TIPS) surgical procedure. TIPS is a procedure to create new connections between the systemic circulation, via the hepatic veins, and the portal venous system. The most common purpose of the TIPS procedure is to decompress the portal venous system by creating a bypass to the systemic veins in the setting of portal hypertension, which is usually caused by liver failure or cirrhosis. When untreated, portal hypertension can cause fluid to build up in the abdomen (“ascites”) and/or chest (“pleural effusion/hepatic hydrothorax”) causing symptoms including risk of infection, require frequent drainage, and can cause increased pressure and subsequent increased size/number of abnormal vessels, called varices, along the upper GI tract which can cause severe GI bleeding. Among other issues, these conditions can cause significant morbidity and mortality. FIG. 6 presents a schematic of anatomy involved in a TIPS procedure that can be performed in accordance with the present disclosure.

During the procedure, the practitioner inserts the catheter through the skin into a vein in the neck of the patient. For example, the catheter can be inserted into the jugular vein of the patient. Using fluoroscopy, the practitioner can guide the catheter into a systemic vein in the liver, specifically, a hepatic vein. Dye or a similar contrast material can be injected into the vein so that it can be seen more clearly. Then, the portal venous system is identified by one of a variety of methods, and a connection is made from the hepatic vein to the portal vein using a needle through the liver. The tract is then dilated, and a stent is placed to keep the connection open. The catheter can be removed.

For TIPS procedures specifically, the system can utilize intra-cardiac echocardiography (ICE) to provide real-time visualization of critical anatomical structures, including the hepatic vein access point and portal vein target. The system facilitates navigation by tracking the position and orientation of the probe in real-time, while simultaneously registering this information with pre-operative imaging. The system can generate 3D/4D visual representations of holograms from ICE or IVUS within a lumen, enabling optimal spatial assessment for clinical decision-making.

In operation and where the probe includes the transesophageal echocardiography (TEE) probe for live 3D TEE, the data collected can include mutually perpendicular images that intersect the heart. For retrospective tomographic data, the 3D sections can include multiplanar sections that longitudinally and transversely intersect the track device or transducer. The 3D set of view or sections can be projected so that each individual view or section can maintain the 3D spatial relationship to each other and to the physical patient. Note that in the case of live 3D TEE or other live imaging, the 3D set of images can be frame grabbed from a 2D screen, arranged in 3D space, and projected in the acquired 3D arrangement.

The 3D views in a 3D spatial arrangement can overlap and obscure each other and/or the virtual representation of the tracked device, such as the probe or the catheter, depending upon the view of the practitioner. To overcome this, a 3D expanded view can be used for stereoscopic projection of 3-dimensional imaging views and the tracked device. Each of the views can be duplicated and translated while maintaining the orientation relative to the centered 3D set and the physical patient. The translation vector can be along the respective normal vector of the imaged section, but other translation vectors can also be used to enable unobscured visualization of the individual sections in 3D projection. The translated views can be cross-referenced with line segments to the 3D set. The 3D expanded view can be applicable to mutually perpendicular planar or section based on thick reprojection slabs centered at point locations of interest during navigation, where the thick slabs can be MIP or Volume rendered reprojections of a 3D image data set

Referring now to registration, live 3D images from the endoluminal probe can be registered with 3D retrospective tomographic images. The 3D CT data set can be registered to the patient using a Vuforia (PTC) AMT or one or more image targets which can be placed relative to a landmark on the patient. For example, three 2D image targets and three points on the feet of the EM sensor in contact with the body/CT skin surface can be tracked relative to a 6 DOF EM sensor. The HMD camera can locate the physical Vuforia component in HMD coordinates, and the Vuforia component can provide the transformation of the 3D CT data set to corresponding physical locations in world coordinate system of the HMD prior to stereoscopic perspective projection with the HMD. A transformation from the tracking coordinates to HMD world coordinates can be provided. Thus, the 3D tracked live and 3D retrospective imaging can be co-registered in physically localizable HMD coordinates and then can be stereoscopically co-projected in correlation with the physical patient in side-by-side or fused 3D Exp views.

It should be appreciated that other optical methods of tracking, such as Hyper Spectral Imaging (HSI) can be implemented. Intraoperative HSI can be applied to tissue recognition and perfusion assessment. Tissue recognition can include: (i) cancer recognition; (ii) anatomical structures recognition; and (iii) thermal ablation efficacy recognition. Perfusion assessment can include: (i) colorectal surgery; (ii) upper gastrointestinal surgery; (iii) hepatopancreaticobiliary surgery; (iv) reconstructive surgery; (v) urology; and (vi) neurosurgery.

Tissue recognition examples further include the following aspects. Gastrointestinal tumor: study using a modified flexible endoscope to recognize colorectal tumors in vivo in human subjects. Deep learning algorithms, in particular neural networks, can promptly differentiate between different tissues, based on their spectral characteristics, either completely automatically (unsupervised learning) or after learning from previously annotated images (supervised learning). Colorectal & Esophageal Cancer: able to achieve an acceptable automating recognition accuracy by using several machine learning supervised methods. Portal Vein, Hepatic Artery, and Common Bile Duct: a laparoscopic HSI camera customized with a spectral scanning (LCTF) HSI system was employed by the authors.

### EXAMPLES

Example embodiments of the present technology are provided with reference to the several figures enclosed herewith.

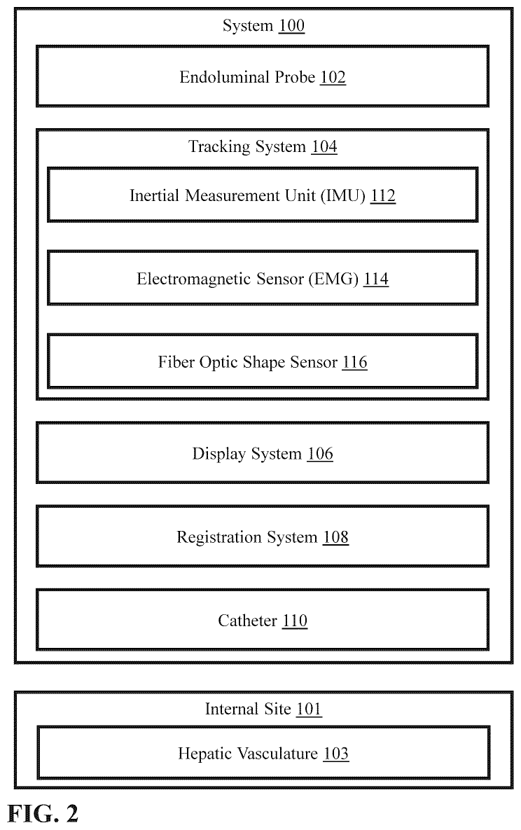

With reference to the accompanying drawings, FIG. 1 is a schematic that describes a system 100, according to some embodiments of the present disclosure. The system 100 can include an endoluminal probe 102 configured to acquire real-time images of an internal site 101, shown in FIG. 2, within the patient, a tracking system 104 configured to track a position and orientation of the endoluminal probe 102, a display system 106 configured to display the real-time images acquired by the endoluminal probe 102, and a registration system 108 configured to register the real-time images from the endoluminal probe 102 with a preoperative image of the internal site 101. The display system 106 can overlay the registered real-time images from the endoluminal probe 102 with the preoperative image to assist in navigation and intervention at the internal site 101.

The endoluminal probe 102 can include an ultrasound transducer located at a distal end configured to ultrasonically image the internal site 101. The tracking system 104 can include advanced model targeting of the endoluminal probe 102 to determine the position and orientation. The advanced model targeting can utilize optical tracking of fiducial markers on the endoluminal probe 102, which can include different geometric shapes or patterns, QR codes, bar codes, and alphanumeric codes.

The markers can be used for registration purposes and can contain important information such as patient identifiers, surgical site details, inventory management data for surgical instruments and implants, and information about the location, position, orientation, alignment and direction of movement of the surgical site and instruments. The advanced model targeting can also use an entire 3D model, but it is also possible to use one or more 2D image targets to create a 3D reference, which can standalone but can also be paired with any of the sensors and sensing methods described herein.

The tracking system 104 can include an inertial measurement unit (IMU) and an EM sensor integrated with the endoluminal probe 102 to track the position and orientation. The endoluminal probe 102 can include an ultrasound transducer located at a distal end configured to ultrasonically image the internal site 101 in three dimensions. The tracking system 104 can be configured to track a position and an orientation the catheter 110 configured to create a shunt between a hepatic vein and a portal vein under image guidance. The display system 106 can be configured to display a projected trajectory of the catheter 110 registered with the real-time IVUS images from the hepatic vasculature 103.

With continued reference to FIG. 1, the tracking system 104 of the system 100 can include an inertial measurement unit (IMU) 112, an electromagnetic (EM) sensor 114, and a fiber optic shape sensor 116. At least one of these sensors can be integrated with the endoluminal probe 102 to track the position and orientation thereof. It should be appreciated that the IMU 112 and the EM sensor 114 can be wireless, as desired. The system 100 can include a catheter 110 configured to be inserted within an anatomy passageway of the patient and the tracking system 104 can be further configured to track a position and an orientation of a catheter 110. The display system 106 can be further configured to display a representation of the catheter 110 registered with the real-time images from the endoluminal probe 102 overlaid on the preoperative image.

The display system 106 can include an extended reality headset configured to stereoscopically display the registered real-time images overlaid on the preoperative image. The preoperative image can be obtained from one of computed tomography (CT), magnetic resonance imaging (MRI), and positron emission tomography (PET) of the patient. The registration system 108 can be configured to dynamically update registration between the real-time images and the preoperative image to account for patient motion.

With continued reference to FIG. 1, the system 100 can include the catheter 110 configured to be inserted in an anatomy passageway of the patient. The catheter 110 can be flexible to better allow the catheter to move along and conform with the anatomy of the patient. As described herein, the tracking system 104 can include the fiber optic sensor 116 and, in certain embodiments, the catheter 110 can be a part of the tracking system 104 and can also include the fiber optic sensor 116. The fiber optic shape sensor 116 of the catheter can track a position and shape of the catheter 110. The display system 106 can be configured to render a computer model of the catheter 110 derived from the fiber optic shape sensing and register the computer model with the real-time endoluminal probe 102 images and the preoperative image.

FIG. 2 is a schematic diagram that further describes the system 100 from FIG. 1, according to some embodiments of the present disclosure. The internal site 101 can include hepatic vasculature 103 accessed via a jugular vein of the patient. The endoluminal probe 102 can include an intravascular ultrasound (IVUS) configured to image the hepatic vasculature 103.

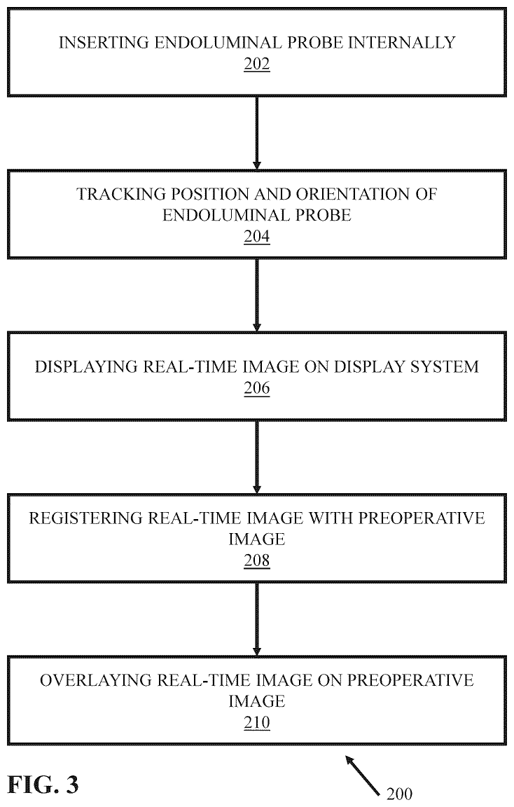

FIG. 3 is a flowchart that describes a method 200, according to one embodiment of the present disclosure. The method 200 can include a step 202 of inserting the endoluminal probe 102 the interior of the body of the patient to acquire real-time images of the internal site 101 within the patient. A position and orientation of the endoluminal probe 102 can be tracked in a step 204. The method 200 can include a step 206 of displaying the real-time images from the endoluminal probe 102 on the display system 106. The real-time images can be registered with a preoperative image of the internal site 101 in a step 208. In a step 210 of the method 200, the registered real-time images can be overlapped on the preoperative image to assist in navigation and intervention of the internal site and displayed in the HMD via the AR system thereby allowing the practitioner to reference and use the display as a guide for performing the procedure. The method 200 can include iterative operations, multiple loops of certain operations, as well as recursive operations, including such embodiments where a single operation, a portion of the operations, or an entirety of the operations depicted in FIG. 3 are iterative, loops, or recursive in nature.

Methods can include updating the registration between the real-time images and the preoperative image dynamically to account for patient motion. Particular aspects can include where inserting the endoluminal probe 102 can include inserting an intravascular ultrasound (IVUS) catheter 110 into a blood vessel of the patient. Likewise, inserting the endoluminal probe 102 can include inserting a transesophageal echocardiogram (TEE) probe into an esophagus of the patient. Inserting the endoluminal probe 102 can include inserting an intracardiac echocardiography (ICE) catheter 110 configured to image a heart of the patient. Inserting the endoluminal probe 102 can include inserting an intravascular ultrasound (IVUS) catheter into a jugular vein and positioning the IVUS catheter in a hepatic vein of the patient.

Aspects of the present methods 200 can relate to transjugular intrahepatic portosystem shunt (TIPS) surgical procedures, which can include tracking a position and an orientation of the catheter 110 inserted through the jugular vein. The method 200 can be configured to place a shunt between the hepatic vein and a portal vein under image guidance, as shown in FIG. 6. Displaying the real-time images can include displaying a projected trajectory of the catheter 110 registered with the real-time IVUS images from the hepatic vein. The preoperative image can include a CT or MRI image depicting hepatic vasculature anatomy.

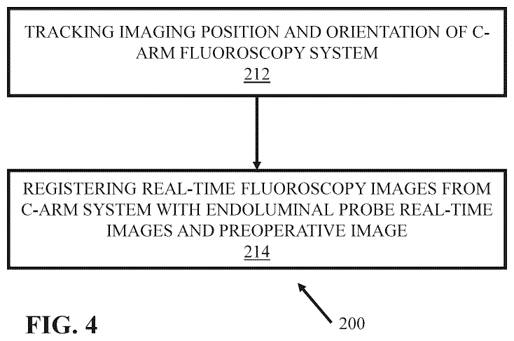

FIG. 4 is a flowchart that further describes the method from FIG. 3, according to one embodiment of the present disclosure which utilizes the system of FIGS. 1 and 2. The method 200 can include a step 212 of tracking an imaging position and orientation of a C-arm fluoroscopy system 118. At a step 214, the tracking can include registering real-time fluoroscopy images from the C-arm system 118 with the endoluminal probe 102 real-time images and preoperative image. As noted for the method 200 of FIG. 3, the operations in FIG. 4 can also be iterative operations, looping operations, as well as recursive operations, including such embodiments where a single operation, a portion of the operations, or an entirety of the operations depicted in FIGS. 3 and 4 are iterative, loops, or recursive in nature. It should also be appreciated that although FIG. 4 details use of real time CT, the method can employ real time ultrasound imaging fused with fluoroscopic imaging.

In one example, the system 100 can be employed to guide a transjugular intrahepatic portosystemic shunt during a TIPS procedures for a patient with portal hypertension, as shown in FIG. 6. The TIPS procedure can use image guidance to place a shunt between the hepatic vein and portal vein within the liver to relieve pressure. An intravascular ultrasound (IVUS) catheter can be inserted through the jugular vein and positioned within a hepatic vein. The IVUS catheter can include an integrated tracking sensor such as the IMU and fiber optic shape sensor to track the position of the catheter.

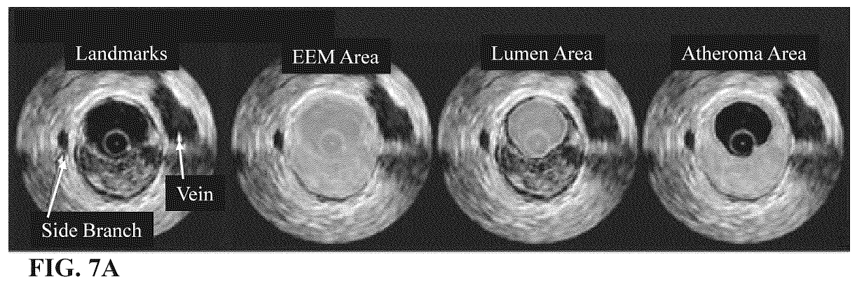

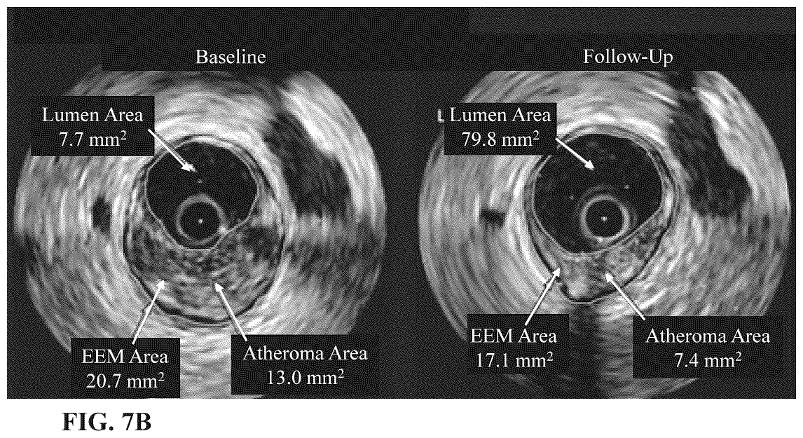

As the IVUS catheter acquires real-time images of the hepatic vasculature, the images can be registered with a preoperative CT scan showing the portal and hepatic vein anatomy. FIGS. 7A and 7B depict images of IVUS for atherosclerosis applications.

Registration is achieved using the tracked position of the IVUS transducer to align the IVUS imaging plane with the corresponding CT slice depicting the surrounding anatomy. The registered, augmented view can be displayed in a stereoscopic augmented reality headset, allowing the practitioner to visualize the internal anatomy in context.

With the target anatomy visualized, the practitioner can introduce a puncture needle to place the shunt between the portal and hepatic veins. The position of the needle can also be tracked using the augmented reality system. The projected trajectory of the needle can be displayed in the headset, registered to the IVUS imaging to enable accurate guidance through the liver parenchyma. The augmented display enhances visualization and spatial understanding throughout the procedure.

In another embodiment, the system 100 can assist with sampling peripheral lung lesions using endobronchial ultrasound guidance. An EBUS catheter with an integrated ultrasound transducer can be inserted through the trachea to image the airway wall and surrounding structures. The EBUS catheter can include an EM sensor and an IMU to localize the position of the EBUS catheter.

As the catheter is navigated towards a lung lesion identified on CT, the real-time EBUS images can be registered to the CT chest scan based on the tracked location and orientation of the transducer, which aligns the ultrasound imaging plane with the corresponding CT slices for intuitive visualization of the lesion anatomy. The augmented EBUS view can be displayed in a stereoscopic headset, fusing CT-derived tumor boundaries with the live ultrasound scan.

With the lesion identified by the registered augmented display, the EBUS catheter is positioned adjacent to the lesion. A biopsy needle is introduced through the catheter channel and deployed into the lesion under continuous ultrasound visualization. The tracking sensors on the needle confirm the projected path of the needle through the lung parenchyma. The system 100 provides enhanced procedural guidance and spatial awareness throughout the minimally invasive lung biopsy.

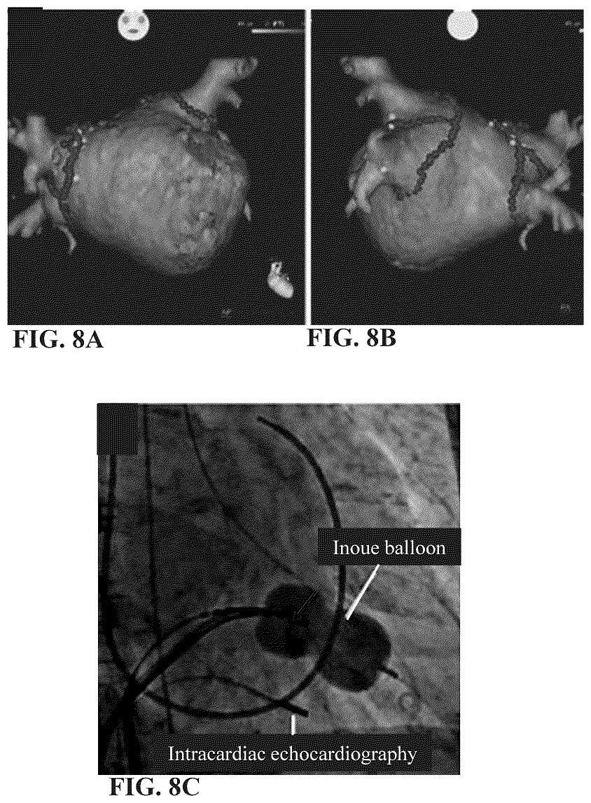

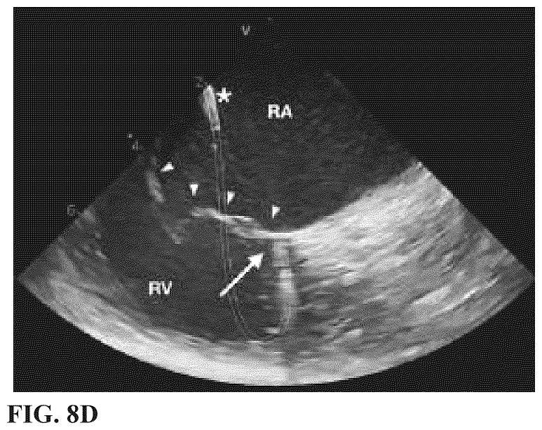

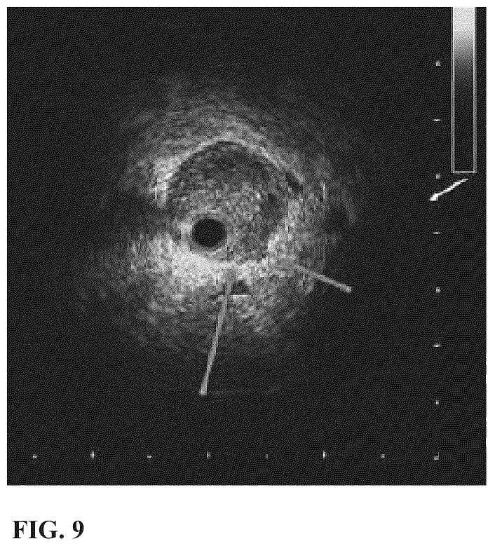

It is important to note that the system 100 can be utilized with intracardiac echocardiograph (ICE) for cardiac ablation applications, as shown in FIGS. 8A-8D, as well as endoluminal ultrasound (ELUS) for urothelial or bladder carcinoma application, as shown in FIG. 9. Cardiovascular heart TEE (transesophageal echocardiography), IVUS, intra-cardiac echocardiography (ICE), optical coherence tomography (OCT) applications can include the following aspects. Certain structural heart cases utilize trans-esophageal echo and/or ICE for guidance to the relevant anatomy. Fluoroscopy can also be used to help with anatomy location and probe orientation. The present technology can serve to minimize fluoroscopy while also improving operator confidence, speed, and navigation, by allowing for direct 3D visualization of the probe 102 and allowing for tool orientation and directionality as well as orientation of relevant surrounding anatomy. Similar to the above TIPS example, stent placement within a coronary stenosis can be visualized along the path of with fiberoptic or wireless EM navigation modalities, visualized in AR, data represented in 3D space, predicted and confirmed stent size and location.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments can be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A system for image-guided intervention of a patient, comprising:
an endoluminal probe configured to acquire a real-time image of an internal site within the patient;
a tracking system configured to track a position and an orientation of the endoluminal probe;
a display system configured to display the real-time image acquired by the endoluminal probe; and
a registration system configured to register the real-time image from the endoluminal probe with a preoperative image of the internal site,
wherein the display system overlays the registered real-time image from the endoluminal probe with the preoperative image to assist in navigation and intervention at the internal site.

2. The system of claim 1, wherein the endoluminal probe includes an ultrasound transducer located at a distal end configured to ultrasonically image the internal site.

3. The system of claim 1, wherein the tracking system includes at least one of:
an inertial measurement unit (IMU);
an electromagnetic sensor; and
a fiber optic shape sensor,
integrated with the endoluminal probe to track the position and the orientation of the endoluminal probe.

4. The system of claim 1, wherein the tracking system includes a wireless inertial sensor and wireless electromagnetic sensor attached to the endoluminal probe.

5. The system of claim 1, wherein the tracking system includes advanced model targeting of the endoluminal probe to determine the position and the orientation.

6. The system of claim 5, wherein the advanced model targeting includes optical tracking of a fiducial marker on the endoluminal probe.

7. The system of claim 1, wherein the display system includes an extended reality headset configured to stereoscopically display the registered real-time image overlaid on the preoperative image.

8. The system of claim 1, wherein the preoperative image is obtained from one of computed tomography (CT), magnetic resonance imaging (MRI), and positron emission tomography (PET) of the patient.

9. The system of claim 1, wherein the registration system is configured to dynamically update registration between the real-time image and the preoperative image to account for patient motion.

10. The system of claim 1, further comprising a catheter configured to be inserted within the patient, wherein the tracking system is further configured to track a position and an orientation of the catheter.

11. The system of claim 10, wherein the display system is further configured to display a representation of the catheter registered with the real-time image from the endoluminal probe overlaid on the preoperative image.

12. The system of claim 1, wherein the endoluminal probe includes an ultrasound transducer located at a distal end configured to ultrasonically image the internal site in three dimensions.

13. The system of claim 1, further comprising a catheter configured to be inserted in an anatomy passageway of the patient, the catheter including a fiber optic shape sensors to track a position and a shape of the catheter.

14. The system of claim 13, wherein the display system is configured to render a computer model of the catheter derived from the fiber optic shape sensing and register the computer model with the real-time endoluminal probe image and the preoperative image.

15. The system of claim 1, wherein the endoluminal probe includes an intravascular ultrasound (IVUS) catheter configured to image a hepatic vasculature of the patient.

16. The system of claim 1, wherein the tracking system is configured to track a position and orientation of a puncture needle configured to place a shunt between a hepatic vein and a portal vein of a hepatic vasculature of the patient under image guidance.

17. The system of claim 16, wherein the display system is configured to display a projected trajectory of the puncture needle registered with a real-time IVUS images from the hepatic vasculature.

18. A method for image-guided intervention of a patient, comprising:
inserting an endoluminal probe internally to acquire a real-time image of an internal site within the patient;
tracking a position and an orientation of the endoluminal probe;
displaying the real-time image from the endoluminal probe on a display system;
registering the real-time image with a preoperative image of the internal site;
overlaying the registered real-time image on the preoperative image to assist in navigation and intervention of the internal site.

19. The method of claim 18, further comprising updating the registration between the real-time images and the preoperative image to account for patient motion.

20. The method of claim 18, further comprising tracking an imaging position and orientation of a C-arm fluoroscopy system; and
registering real-time fluoroscopy images from the C-arm system with the endoluminal probe real-time image and preoperative image.
